(19) United States
(12) Patent Application Publication
(10) Pub. No.: US 2012/0302640 A1
(43) Pub. Date: Nov. 29, 2012

(54) ORAL FORMULATIONS

(76) Inventor: Don Macalister, Herne Bay (NZ)

(21) Appl. No.: 13/524,646

(22) Filed: Jun. 15, 2012

Related U.S. Application Data
(63) Continuation-in-part of application No. PCT/NZ2010/000256, filed on Dec. 17, 2010.

(30) Foreign Application Priority Data
Dec. 17, 2009 (NZ) 582165

(51) Int. Cl. A61K 31/195; A61P 31/04; A61P 29/00; A61P 7/04

(52) U.S. Cl. 514/561

(57) ABSTRACT

The invention relates to a composition, in the form of a gel, comprising tranexamic acid for topical oral application to a tooth extraction site either before, during or after the tooth extraction to control dental bleeding, to treat or prevent alveolar osteitis, or to produce or maintain a blood clot. The composition may comprise additional components such as chlorhexidine, methyl cellulose, a sweetener, a flavouring agent (e.g. mint), and a preservative (e.g. potassium sorbate).

### RELATED APPLICATIONS AND INCORPORATION BY REFERENCE

This application is a continuation-in-part application of international patent application Serial No. PCT/NZ2010/000256 filed 17 Dec. 2010, which published as PCT Publication No. WO 2011/078700 on 30 Jun. 2011, which claims benefit of New Zealand patent application Serial No. 582165 filed 17 Dec. 2009.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to oral formulations that may contain tranexamic acid and their uses, such as to treat or prevent alveolar osteitis.

### BACKGROUND OF INVENTION

A most common complication worldwide after the extraction of a tooth is the condition variously known as alveolar osteitis, fibrinolytic osteitis, post operative bone inflammation or, as it is most commonly known, “dry socket”.

It is a painful and distressing post operative condition identified pathognomically as a dull throbbing pain presenting in and around the extraction site between one to three days after the extraction.

It is often accompanied by the visible breakdown of the blood clot associated with the socket.

Its etiology is largely unknown but it is widely hypothesized that the main factors are the failure to form or retain a satisfactory organized blood clot in the extraction socket. Failure of the clot leads to a failure of healing with its attendant discomfort.

This failure to produce an organized blood clot has been ascribed to an initial failure to clot and form a stabilized clot or the subsequent early breakdown of the clot due to the lytic effect of bacterial enzymes attacking the clot at the site.

There is a growing demographic worldwide for an aging population that is living longer and remaining dentate late into life, is on anti coagulant therapy and needs teeth extracted on occasion.

A number of attempts have been made to treat “dry socket”.

U.S. Pat. No. 7,517,539 relates to the use of an implant that contains bioresorbable and reossifyling PCA calcium phosphate as an implantable bioceramic. It can be applied to bone defects as a filler and it promotes the growth of new bone tissue. It also reports that application of the implant to a tooth socket can avoid dry socket.

U.S. Pat. No. 7,157,614 relates to the use of an antimicrobial sheet having a first layer that includes a block copolymer and mineral oil and a second layer that contains a bactericidal metal. This patent reports that the device has been found to prevent dry socket and to promote rapid healing.

U.S. Pat. No. 6,146,655 relates to the use of a kit for preparing a flexible intra-oral bandage that is formed from a water permeable envelope of non-woven water porous fabric and a powder/fibre mixture contained within the envelope, and also a particulate water-soluble powder that contains soluble alginate salt to prevent or treat dry socket.

U.S. Pat. No. 5,006,071 relates to the use of a mouldable, absorbable composition that is formed from plaster of Paris with tetracycline and hydrocortisone that prevents dry socket.

U.S. Pat. No. 4,677,139 relates to the use of a foam dressing that prevents dry socket. The foam dressing is a biocompatible silicone elastomeric foam composition.

U.S. Pat. No. 4,357,935 relates to the use of a dressing for treating dry socket. The dressing includes a gauze strip that is impregnated with a gelatinous material that contains tetroliten emulsifiers and an analgesic substance.

US patent application 2007/0190110 relates to the use of unwoven fibre material that is a cellulose based non-synthetic material that is oxidised and can be absorbed into biological tissue for treating dry socket.

US patent application 2007/0014862 relates to the use of a haemostatic agent that comprises oxidised cellulose that can be shaped and placed into a bleed site or into a wound gap. Dry socket is given as an example of a condition that can be treated.

US patent application 2006/0089584 relates to the use of a dental pad for avoiding dry socket. The dental pad is formed of a hydrophilic polymer sponge that includes a chitosan biomaterial.

US patent application 2006/0062834 relates to the use of a wound care dressing that comprises a non woven blanket that comprises a sacrilyde for treating dry socket.

US patent application 2005/0036955 relates to the a method for preventing dry socket by filling the oral cavity with a biocompatible, bioabsorbable dressing that is prepared by reacting a collagen derivative and cytotoxic crosslinking agent. Examples of collagen derivatives include gelatine. An example of a crosslinking agent includes metal cations, peroxides and mixtures thereof.

None of the above approaches are particularly effective, nor do they provide an effective solution for patients that suffer from a bleeding disorder, or are using anticoagulant medications such as aspirin or warfarin.

Citation or identification of any document in this application is not an admission that such document is available as prior art to the present invention.

### SUMMARY OF THE INVENTION

An object of the present invention is to provide an oral formulation in the form of a gel that may contain tranexamic acid able to prevent, ameliorate and/or treat any one or more of the aforementioned conditions, complications or concerns, or to at least provide the public with a useful choice.

Accordingly, in a first aspect the present invention relates to a composition, in the form of a gel, may comprise tranexamic acid.

Another aspect of the invention relates to a composition, in the form of a gel, for topical oral application, the gel may comprise tranexamic acid to
control dental bleeding,
treat or prevent alveolar osteitis, or
produce or maintain a blood clot in a tooth extraction site of a patient.

Another aspect of the invention relates to the use of tranexamic acid in the manufacture of a gel for controlling dental bleeding.

Another aspect of the invention relates to a method for controlling dental bleeding in a subject in need thereof after tooth extraction which may comprise topical administration of an effective amount of a composition, in the form of a gel, that may comprise tranexamic acid to the tooth extraction site.

Another aspect of the invention relates to a composition, in the form of a gel, that may comprise tranexamic acid for use in controlling dental bleeding.

Another aspect of the invention relates to the use of tranexamic acid in the manufacture of a gel to treat or prevent alveolar osteitis.

Another aspect of the invention relates to a method for treating or preventing osteitis in a subject in need thereof after tooth extraction which may comprise topical administration of an effective amount of a composition, in the form of a gel, that may comprise tranexamic acid to the tooth extraction site.

Another aspect of the invention relates to a composition, in the form of a gel, that may comprise tranexamic acid for use in treating or preventing alveolar osteitis.

Another aspect of the invention relates to the use of tranexamic acid in the manufacture of a gel for producing or maintaining a blood clot in a tooth extraction site in a patient to which an anticoagulant has been, or is to be, administered, or who suffers from an excessive bleeding disorder.

Another aspect of the invention relates to a method for producing or maintaining a blood clot in a tooth extraction site in a subject in need thereof to which an anticoagulant has been, or is to be, administered, or who suffers from an excessive bleeding disorder, which may comprise topical administration of an effective amount of a composition, in the form of a gel, that comprises tranexamic acid to the tooth extraction site.

Another aspect of the invention relates to a composition, in the form of a gel, that may comprise tranexamic acid for use in producing or maintaining a blood clot in a tooth extraction site in a patient to which an anticoagulant has been, or is to be, administered, or who suffers from an excessive bleeding disorder.

Another aspect of the invention relates to the use of tranexamic acid and an antibacterial compound (e.g. chlorhexidine), together with carrier materials, in the manufacture of an oral gel composition, suitable to treat or address any of the conditions previously mentioned.

Another aspect of the invention relates to a topical oral composition in a suitable carrier suitable for application pre, during and/or post extraction to a dental extraction site, the composition which may have both tranexamic acid and chlorhexidine.

Another aspect of the invention relates to a composition in gel form able to be applied topically to a dental extraction site with a view to obviating and/or treating the conditions variously known as alveolar osteitis, fibrinolytic osteitis, postoperative bone information or dry socket, the composition having, in the carrier that provides the gel form, an effective amount of tranexamic acid as a clot stabiliser and an effective amount of chlorhexidine as an antibacterial active.

Another aspect of the invention relates to a composition in a gel formulation able to be applied topically to a dental extraction site with a view to obviating and/or treating the condition variously known as alveolar osteitis, fibrinolytic osteitis, postoperative bone information or dry socket, the formulation which may have an effective amount of tranexamic acid as a clot stabiliser and an effective amount of chlorhexidine as an antibacterial active.

The following embodiments may relate to any of the above aspects.

In some embodiments the composition may be used to treat or prevent alveolar osteitis.

The gel is formulated for topical oral application to a tooth extraction site either before, during or after the tooth extraction.

In some embodiments the gel composition may be applied before, during or after tooth extraction to the dental extraction site.

In some embodiments the gel may be a polysaccharide gel, a cellulosic gel, a methyl cellulosic gel, a hydrogel, agarose, or gelatine.

In some embodiments the composition may comprise about 5-15% by weight tranexamic acid.

In some embodiments the composition may comprise about 5, 6, 7, 8, 9, 10, 11, 12, 13 or 15% by weight tranexamic acid.

In some embodiments the composition may comprise about 1, 5, 10, 15, 20, 25, 30, 35, 40, 45, 50, 55, 60, 65 or 70% by weight tranexamic acid.

Preferably the tranexamic acid may be present at about 10% by weight.

In some embodiments the composition may comprise an antibacterial active.

In some embodiments the composition is formed as a gel which may comprise
about 5 to about 15% by weight tranexamic acid and about 0.05 to about 0.2% by weight of an antibacterial active,
about 5 to about 10% by weight tranexamic acid and about 0.05 to about 0.1% by weight of an antibacterial active,
about 10 to about 15% by weight tranexamic acid and about 0.05 to about 0.1% by weight of an antibacterial active,
about 5 to about 10% by weight tranexamic acid and about 0.1 to about 0.2% by weight of an antibacterial active, or
about 10 to about 15% by weight tranexamic acid and about 0.1 to about 0.2% by weight of an antibacterial active.

In some embodiments the composition may comprise about 0.05, 0.06, 0.07, 0.08, 0.09, 0.1, 0.11, 0.12, 0.13, 0.14, 0.15, 0.16, 0.17, 0.18, 0.19 or 2% by weight of an antibacterial active.

In some embodiments the composition may comprise about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 or 10% by weight of an antibacterial active.

In some embodiments the antibacterial active may be chlorhexidine.

In some embodiments the composition may comprise about 0.05-2% by weight chlorhexidine.

Preferably the chlorhexidine may be present at about 0.15% by weight.

In some embodiments the composition additionally may comprise any one or more of the following:
chlorhexidine,
methyl cellulose,
a sweetener,
a flavouring agent (e.g. mint), and
a preservative (e.g. potassium sorbate).

In some embodiments the extraction site may be exposed to tranexamic acid for at least about 4, 6, 8, 10, 12, 14, 16, 18, 20, 22, 24, 26, 28, 30, 32, 34, 36, 38, 40, 42, 44, 46, 48 or 50 minutes.

In some embodiments about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 or 1 mL composition may be applied to the extraction socket.

In some embodiments the composition may be added within 1, 2, 3, 4, 5, 6, 7, 8, 9, 10, 15, 20, 25, 30, 35, 40, 45, 50 or 60 min after tooth extraction.

In some embodiments about 1, 2, 3, 4 or 5 topical applications of the composition may be applied to the tooth extraction site.

In some embodiments wherein the viscosity of the composition may be about 100, 200, 300, 400, 500, 600, 700, 800, 900, 1000, 2000, 3000, 4000, 5000, 6000, 7000, 8000, 9000, 10,000, 11,000, 12,000, 13,000, 14,000 or 15,000 cP.

In some embodiments the tranexamic acid may be in a dental pad.

In one embodiment a composition useful herein further may comprise a pharmaceutically acceptable carrier.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

To those skilled in the art to which the invention relates, many changes in construction and widely differing embodiments and applications of the invention will suggest themselves without departing from the scope of the invention as defined in the appended claims The disclosures and the descriptions herein are purely illustrative and are not intended to be in any sense limiting.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION OF THE INVENTION

The invention relates the use of compositions comprising tranexamic acid, wherein the composition is a gel and is suitable for topical oral application to a tooth extraction site either before, during or after the tooth extraction. Suitable compositions for use in the invention are described below.

The term “antibacterial active” includes any suitable and compatible active to tranexamic acid that has an antibacterial activity. Chlorhexidine is just one of many such actives.

The term “cellulose derivative gel” is to be interpreted broadly. For instance, to include any methyl cellulose type gel vehicle.

An “effective amount” is the amount required to confer therapeutic effect. The interrelationship of dosages for animals and humans (based on milligrams per meter squared of body surface) is described by Freireich, et al. (1966). Body surface area can be approximately determined from height and weight of the subject. See, e.g., Scientific Tables, Geigy Pharmaceuticals, Ardley, N.Y., 1970, 537. Effective doses also vary, as recognized by those skilled in the art, dependent on route of administration, carrier usage, and the like.

The term “gel” is not confined to methyl cellulose type gels. Orally acceptable agarose, gelatine, and other gel vehicles can be used.

The term “methyl cellulose” in respect of gels can include, for example, HPMC or HEMC.

The term “pharmaceutically acceptable carrier” includes excipients, diluents, auxiliaries, and combinations thereof selected with regard to the intended route of administration and standard pharmaceutical practice. See for example, Remington's Pharmaceutical Sciences, 16th edition, Osol, A. Ed., Mack Publishing Co., 1980.

The term “preservative” includes any suitable preservative agent e.g. sodium benzoate, potassium sorbate, etc.

A “subject” is an animal, preferably a mammal. In one embodiment the subject is a human. Preferably the human is an adult, a child, or an infant.

The term “sweetener” preferably is non-caloric but need not be. Examples of sweetener restricted to aspartame, saccharin and acesulfame K.

The term “treat” and its derivatives should be interpreted in their broadest possible context. The term should not be taken to imply that a subject is treated until total recovery. Accordingly, “treat” broadly includes amelioration and/or prevention of the onset of the symptoms or severity of a particular condition.

The term “tranexamic acid” includes the acid itself or bioprecursors or salts thereof having the desired hemostatic activity.

The invention generally relates to the application of a gel formulation, able to be applied topically, to a dental extraction site with a view to obviating and/or treating alveolar osteitis, the formulation having an effective amount of tranexamic acid.

The invention also relates to the application of a gel formulation, able to be applied topically, to a dental extraction site in patients to which an anticoagulant has been, or is to be, administered, or who suffers from an excessive bleeding disorder.

Tranexamic acid [trans-4-(aminomethyl)cyclohexanecarboxylic acid, CAS number 1197-18-8] is an antifibrinolytic that competitively inhibits the activation of plasminogen to plasmin.

In some embodiments the gel is a polysaccharide gel, a cellulosic gel, a methyl cellulosic gel, a hydrogel, agarose, gelatine, or any other suitable gel vehicles.

In some embodiments the composition comprises about 5, 6, 7, 8, 9, 10, 11, 12, 13 or 15% by weight tranexamic acid and useful ranges may be selected between any of these values (for example, about 5 to about 15, from about 5 to about 13, from about 5 to about 11, from about 5 to about 10, from about 5 to about 8, from about 7 to about 15, from about 7 to about 10, from about 8 to about 15, from about 8 to about 13, from about 8 to about 10% by weight tranexamic acid).

In some embodiments the composition comprises about 1, 5, 10, 15, 20, 25, 30, 35, 40, 45, 50, 55, 60, 65 or 70% by weight tranexamic acid and useful ranges may be selected between any of these values (for example, about 1 to about 70, about 1 to about 55, about 1 to about 30, about 1 to about 10, about 1 to about 5, about 5 to about 70, about 5 to about 60, about 5 to about 15, about 5 to about 10, about 10 to about 15, about 10 to about 30, about 10 to about 40, about 10 to about 55, about 10 to about 70, about 25 to about 70, about 25 to about 50, about 25 to about 45, about 25 to about 30, about 45 to about 70, about 45 to about 60, about 45 to about 50, about 50 to about 70 or about 55 to about 70% by weight tranexamic acid).

In some embodiments the tranexamic acid is present at about 10% by weight.

In some embodiments the composition comprises an antibacterial active.

In some embodiments the composition is formed as a gel and comprises
about 5 to about 15% by weight tranexamic acid and about 0.05 to about 0.2% by weight of an antibacterial active,
about 5 to about 10% by weight tranexamic acid and about 0.05 to about 0.1% by weight of an antibacterial active,
about 10 to about 15% by weight tranexamic acid and about 0.05 to about 0.1% by weight of an antibacterial active,
about 5 to about 10% by weight tranexamic acid and about 0.1 to about 0.2% by weight of an antibacterial active, or
about 10 to about 15% by weight tranexamic acid and about 0.1 to about 0.2% by weight of an antibacterial active.

In some embodiments the composition comprises about 0.05, 0.06, 0.07, 0.08, 0.09, 0.1, 0.11, 0.12, 0.13, 0.14, 0.15, 0.16, 0.17, 0.18, 0.19 or 2% by weight of an antibacterial active and useful ranges may be selected between any of these values (for example, about 0.05 to about 2, from about 0.05 to about 0.16, from about 0.05 to about 0.13, from about 0.05 to about 0.1, from about 0.05 to about 0.08, from about 0.07 to about 0.2, from about 0.07 to about 0.18, from about 0.07 to about 0.15, from about 0.07 to about 0.11, from about 0.07 to about 0.1, from about 0.09 to about 2, from about 0.09 to about 0.15, from about 0.09 to about 0.12, from about 0.1 to about 0.2, from about 0.1 to about 0.17, from about 0.1 to about 0.15, from about 0.12 to about 0.2, from about 0.12 to about 0.15, from about 0.15 to about 0.2% by weight of an antibacterial active).

In some embodiments the composition comprises about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 or 10% by weight of an antibacterial active and useful ranges may be selected between any of these values (for example, about 0.01 to about 10, about 0.01 to about 5, about 0.01 to about 2, about 0.01 to about 1, about 0.01 to about 0.5, about 0.01 to about 0.1, about 0.01 to about 0.05, about 0.05 to about 10, about 0.05 to about 4, about 0.05 to about 2, about 0.05 to about 1, about 0.05 to about 0.5, about 0.05 to about 0.1, about 0.1 to about 10, about 0.1 to about 5, about 0.1 to about 2, about 0.1 to about 1, about 0.1 to about 0.5, about 0.4 to about 10, about 0.4 to about 6, about 0.4 to about 2, about 0.4 to about 1, about 0.4 to about 0.8, about 1 to about 10, about 1 to about 8, about 1 to about 4, about 1 to about 2, about 2 to about 10, about 2 to about 5 or about 5 to about 10% by weight of an antibacterial active).

In some embodiments the antibacterial active is chlorhexidine.

In some embodiments the chlorhexidine is present at about 0.15% by weight.

In some embodiments the composition additionally comprises any one or more of the following:
chlorhexidine,
methyl cellulose,
a sweetener,
a flavouring agent (e.g. mint), and
a preservative (e.g. potassium sorbate).

In some embodiments the viscosity of the composition is about 100, 200, 300, 400, 500, 600, 700, 800, 900, 1000, 2000, 3000, 4000, 5000, 6000, 7000, 8000, 9000, 10,000, 11,000, 12,000, 13,000, 14,000 or 15,000 cP and useful ranges may be selected between any of these values (for example, about 100 to about 15,000, about 100 to about 13,000, about 100 to about 10,000, about 100 to about 8000, about 100 to about 5,000, about 100 to about 3,000, about 100 to about 900, about 100 to about 500, about 400 to about 15,000, about 400 to about 12,000, about 400 to about 10,000, about 400 to about 8,000, about 400 to about 5,000, about 400 to about 3,000, about 400 to about 1,000, about 400 to about 800, about 400 to about 600, about 900 to about 15,000, about 900 to about 12,000, about 900 to about 9,000, about 900 to about 4,000, about 900 to about 2,000, about 2,000 to about 15,000, about 2,000 to about 11,000, about 2,000 to about 9,000, about 2,000 to about 6,000, about 2,000 to about 4,000, about 4,000 to about 12,000, about 4,000 to about 7,000, about 4000 to about 5,000, about 8,000 to about 15,000, about 8,000 to about 11,000, about 11,000 to about 15,000 or about 11,000 to about 12,000 cP).

In some embodiments the tranexamic acid gel is on a dental pad.

In one embodiment a composition useful herein further comprises a pharmaceutically acceptable carrier.

It should be appreciated that the gel is formulated with a certain viscosity and adherence to remain in the extraction site.

A preferred composition in accordance with the present invention is a topical oral gel containing in any suitable quantities which does not affect its ability to be applied as a gel to a desired locus of activity, a sweetener, some mint flavour, potassium sorbate as preservative, some water and methyl cellulose. The active ingredients present are tranexamic acid at 10% by weight and chlorhexidine at about 0.15% by weight.

The gel can be applied by any suitable means including those discussed previously.

The gel formulation of preferred forms of the present invention preferably is optimized for the oral environment to maximize the effect of the tranexamic acid on external clot initiation and stabilization. The formulation of the invention achieves this much more simply and cost effectively than other presentations and therapies.

The chlorhexidine component of the gel optimizes the conditions conducive to healing and reduces the bacterial attack on the clot.

Any suitable oral gel can be used in the composition and methods of the invention. It should be appreciated that appropriate oral gels can be made by a skilled person with regard to their skill and knowledge and with reference to this specification. In one embodiment, a pre-made gel composition is mixed with an effective amount of tranexamic acid. In another embodiment, the gel components and tranexamic acid can be mixed to form the gel.

The formulation of the present invention enhances healing and decreases the incidence of the most common and distressing post operative complication after tooth extraction, as well as enabling the treatment by dental practitioners of their patients who are on anticoagulant therapies or suffer from a disorder of excessive bleeding, and require tooth extraction.

Causes of excessive bleeding include inherited disorders such as von Willebrand's disease and haemophilia, disorders developed during certain illnesses (such as vitamin K deficiency, severe liver disease, bone marrow problems and pregnancy-associated eclampsia), or treatments (such as use of anticoagulant drugs such as aspirin, heparin, warfarin, and drugs used to break up blood clots or prolonged use of antibiotics).

Tranexamic acid in gel delivery form (alone and preferably with an antibacterial agent, such as chlorhexidine) can assist in stabilizing the extraction site blood clot. Without wishing to be tied to any theory, a gel of tranexamic acid and chlorhexidine acts synergistically to promote the initialization and stabilization of the extraction site blood clot.

The tranexamic acid, in gel delivery form, produces a potent external trigger effect to initiate and stabilize the clot in the socket through the critical immediate post extraction phase.

The inclusion of, for example, chlorhexidine simultaneously reduces the bacterial contamination of the site to aid clot stability and longevity.

Maximizing initial clot stability produces a lower incidence of dry socket, particularly in high risk groups.

Another use for the formulations of the invention is in the treatment of patients with bleeding diathesis or more commonly warfarinised patients who require tooth extraction. Their medical condition often requires that they should not cease their anticoagulant therapy.

Treatment protocols for such patients rely on ascertaining that the patients International Normalised Ratio (measures the time it takes for blood to clot and compares it to an average) is within a certain therapeutic range compatible with haemostasis after extraction, and then the formulation of the invention is applied to the fresh extraction site under a pressure pad.

In some embodiments about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 or 1 mL of composition is applied to the extraction socket minutes and useful ranges may be selected between any of these values (for example, about 0.05 to about 1, about 0.05 to about 0.5, about 0.05 to about 0.1, about 0.1 to about 1, about 0.1 to about 0.8, about 0.1 to about 0.5, about 0.2 to about 1, about 0.2 to about 0.75, about 0.2 to about 0.5, about 0.4 to about 1, about 0.4 to about 0.85, about 0.4 to about 0.3, about 0.8 to about 1 mL of composition).

In some embodiments the extraction site is exposed to tranexamic acid for at least about 4, 6, 8, 10, 12, 14, 16, 18, 20, 22, 24, 26, 28, 30, 32, 34, 36, 38, 40, 42, 44, 46, 48 or 50 minutes and useful ranges may be selected between any of these values (for example, about 4 to about 50, about 4 to about 42, about 4 to about 30, about 8 to about 50, about 8 to about 40, about 8 to about 30, about 12 to about 50, about 12 to about 44, about 12 to about 26, about 16 to about 50, about 16 to about 46, about 16 to about 40, about 16 to about 32, about 20 to about 50, about 20 to about 46, about 20 to about 40, about 20 to about 30, about 26 to about 50, about 26 to about 46, about 26 to about 42, about 26 to about 40, about 26 to about 34, about 30 to about 50, about 30 to about 44, about 30 to about 40, about 32 to about 50, about 32 to about 44, about 32 to about 40, about 40 to about 50 or about 40 to about 44 minutes).

In some embodiments the composition is added within 1, 2, 3, 4, 5, 6, 7, 8, 9, 10, 15, 20, 25, 30, 35, 40, 45, 50 or 60 min after tooth extraction and useful ranges may be selected between any of these values (for example, about 1 to about 60, about 1 to about 50, about 1 to about 30, about 1 to about 15, about 1 to about 10, about 1 to about 5, about 3 to about 60, about 3 to about 40, about 3 to about 30, about 3 to about 15, about 3 to about 10, about 9 to about 60, about 9 to about 50, about 9 to about 30, about 9 to about 20, about 15 to about 60, about 15 to about 45, about 15 to about 35 or about 20 to about 60 min after tooth extraction).

In some embodiments about 1, 2, 3, 4 or 5 topical applications of the composition is applied to the tooth extraction site and useful ranges may be selected between any of these values (for example, about 1 to about 5, about 1 to about 4, about 1 to about 3, about 1 to about 2, about 2 to about 5, about 2 to about 4, about 2 to about 3, about 3 to about 5, about 3 to about 4 or about 4 to about 5 topical applications).

The tranexamic acid gel of the present invention allows for a clean, accurate clot-on-demand.

Without the use of the tranexamic acid gel there would be a lot of bleeding. Furthermore, those patients taking anticoagulation therapies can be treated using the tranexamic acid gel to ensure a tooth extraction as atraumatically as possible.

Techniques used in the past, such as crushing up tablets or soaking swabs did not provide a consistent effect, were hard to standardise, and were not user-friendly.

The advantage of using the tranexamic acid gel is that it is easy to use, provides a quantifiable dose, is sterile and fresh for every patient, requires minimal handling, can be precisely applied to the extraction site, and controls its effect to stabilise the clot being worked with.

These advantages contribute to the efficacy and safety of the tranexamic acid gel process.

Another advantage of the tranexamic acid gel process is when installing dentures immediately after multiple tooth extractions. Even with normal hematology, there is a lot of bleeding, which is difficult to control as the new denture is placed over the wounds at the time the teeth are taken out. By adding the tranexamic acid gel inside the immediate denture and then placing it, you get a very effective hemostasis inside the denture.

In some embodiments the gel provides the exposure of the extraction site to tranexamic acid longer than other oral delivery systems.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Example

A tranexamic acid gel was prepared in accordance with the formulation shown below in Table 1.

TABLE 1
Composition of a gel of the present invention

| Ingredient | % by weight |
|---|---|
| micronised tranexamic acid | 10 |
| chlorhexidine | 0.15 |
| methocell 4M | made up to |
| potassium sorbate | 100% |
| benzyl alcohol | |
| crème de menthe | |
| purified water. | |

A warfarinised patient presents requiring a tooth extraction.

Following removal of the tooth the socket is sutured and tranexamic acid gel is applied to the tooth extraction site using a syringe and ⅙ mm needle to produce a fine gel bead approximately the size of the tip of a biro pen. The tranexamic acid gel is applied to achieve coverage of the bleeding gingiva and the blood that is sitting in the socket.

Following application of the gel a sterile gauze swab is applied on top of the gel coated socket.

The gel remains in place for 30-40 minutes and is effective in maintaining a good blood clot in the extraction socket, and in reducing or preventing the occurrence of alveolar osteitis.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A topical oral formulation comprising, in the form of a gel, 1 to about 70% by weight tranexamic acid, an antibacterial agent and having a viscosity of about 100 to about 15,000 cP.

2. A composition of claim 1 wherein the gel is a polysaccharide gel, a cellulosic gel, a methyl cellulosic gel, a hydrogel, agarose, or gelatine or a combination of any two or more thereof.

3. A composition of claim 1 comprising about 0.05, 0.06, 0.07, 0.08, 0.09, 0.1, 0.11, 0.12, 0.13, 0.14, 0.15, 0.16, 0.17, 0.18, 0.19 or 2% by weight of an antibacterial agent.

4. A composition of claim 1 comprising
about 5 to about 15% by weight tranexamic acid and about 0.05 to about 0.2% by weight of an antibacterial agent,
about 5 to about 10% by weight tranexamic acid and about 0.05 to about 0.1% by weight of an antibacterial agent,
about 10 to about 15% by weight tranexamic acid and about 0.05 to about 0.1% by weight of an antibacterial agent,
about 5 to about 10% by weight tranexamic acid and about 0.1 to about 0.2% by weight of an antibacterial agent, or
about 10 to about 15% by weight tranexamic acid and about 0.1 to about 0.2% by weight of an antibacterial agent.

5. A composition of claim 1 wherein the antibacterial agent is chlorhexidine.

6. A composition of claim 1 wherein the composition additionally comprises any one or more of the following:
methyl cellulose,
a sweetener,
a flavouring agent (e.g. mint), and
a preservative (e.g. potassium sorbate).

7. A method of treating or preventing alveolar osteitis comprising administering a composition, in the form of a gel, that comprises tranexamic acid.

8. A method of producing or maintaining a blood clot in a tooth extraction site in a patient to which an anticoagulant has been, or is to be, administered, or who suffers from an excessive bleeding disorder, comprising administering a composition, in the form of a gel, that comprises tranexamic acid to the tooth extraction site.

9. A method to treat or prevent alveolar osteitis in a subject in need thereof after tooth extraction comprising topical administration of an effective amount of a composition, in the form of a gel, that comprises tranexamic acid to the tooth extraction site.

10. A method of claim 7 wherein the composition comprises 1 to about 70% by weight tranexamic acid, an antibacterial agent and has a viscosity of about 100 to about 15,000 cP.

11. A method of claim 7 wherein the gel is a polysaccharide gel, a cellulosic gel, a methyl cellulosic gel, a hydrogel, agarose, or gelatine or a combination of any two or more thereof.

12. A method of claim 7 wherein the composition comprises about 0.05, 0.06, 0.07, 0.08, 0.09, 0.1, 0.11, 0.12, 0.13, 0.14, 0.15, 0.16, 0.17, 0.18, 0.19 or 2% by weight of an antibacterial agent.

13. A method of claim 7 wherein the composition comprises
about 5 to about 15% by weight tranexamic acid and about 0.05 to about 0.2% by weight of an antibacterial agent,
about 5 to about 10% by weight tranexamic acid and about 0.05 to about 0.1% by weight of an antibacterial agent,
about 10 to about 15% by weight tranexamic acid and about 0.05 to about 0.1% by weight of an antibacterial agent,
about 5 to about 10% by weight tranexamic acid and about 0.1 to about 0.2% by weight of an antibacterial agent, or
about 10 to about 15% by weight tranexamic acid and about 0.1 to about 0.2% by weight of an antibacterial agent.

14. A method of claim 7 wherein the composition comprises chlorhexidine.

15. A method of claim 7 wherein the composition comprises any one or more of the following:
methyl cellulose,
a sweetener,
a flavouring agent (e.g. mint), and
a preservative (e.g. potassium sorbate).

16. A method of claim 7 wherein the extraction site is exposed to tranexamic acid for at least about 4, 6, 8, 10, 12, 14, 16, 18, 20, 22, 24, 26, 28, 30, 32, 34, 36, 38, 40, 42, 44, 46, 48 or 50 minutes.

17. A method of claim 7 wherein 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 or 1 mL composition is applied to the extraction socket.

18. A method of claim 7 wherein the composition is added within 1, 2, 3, 4, 5, 6, 7, 8, 9, 10, 15, 20, 25, 30, 35, 40, 45, 50 or 60 min after tooth extraction.

19. A method of claim 7 wherein 1, 2, 3, 4 or 5 topical applications of the composition is applied to the tooth extraction site.
